(19) United States
(12) Patent Application Publication
(10) Pub. No.: US 2026/0132248 A1
(43) Pub. Date: May 14, 2026

(54) PROCESS AND APPARATUS OF PRODUCTION OF AROMATIC DICARBOXYLIC ACID

(71) Applicant: INEOS US CHEMICALS COMPANY, Wilmington, DE (US)

(72) Inventors: Sean WUNDROW, Naperville, IL (US); Bob WINTERS, Oud-Turnhout (BE); Dominique GEMOETS, Lier (BE)

(21) Appl. No.: 18/849,651

(22) PCT Filed: Mar. 23, 2023
(86) PCT No.: PCT/US2023/064845

§ 371 Date: Sep. 23, 2024

Related U.S. Application Data
(60) Provisional application No. 63/323,208, filed on Mar. 24, 2022.

(30) Foreign Application Priority Data
Apr. 6, 2022 (GB) 2205016.5

(51) Int. Cl. C08G 63/54; B01D 9/00; B01D 53/14; B01J 19/24; C07C 51/16; C07C 51/43

(52) U.S. Cl. CPC C08G 63/54; B01D 9/0059; B01D 53/1418; B01D 53/1487; B01J 19/2445; C07C 51/16; C07C 51/43; B01D 2009/0086; B01D 2257/708; B01D 2258/02

(57) ABSTRACT

A process for production of both purified recycled aromatic dicarboxylic acid (rCA) and purified aromatic dicarboxylic acid (vCA), said process comprising:
Producing a first stream comprising a recycled aromatic dicarboxylic acid by a first process, wherein the first process comprises the depolymerisation of a polymer feedstock comprising a polymer of said aromatic dicarboxylic acid in a first reaction system;
Producing a second stream comprising the aromatic dicarboxylic acid by a second process wherein the second process comprises the oxidation of an aromatic hydrocarbon in a second reaction system;
Purifying both the first stream and the second stream in a common purification system to thereby produce both purified recycled aromatic dicarboxylic acid (rCA) and purified aromatic dicarboxylic acid (vCA).

The present invention also provides an apparatus for the production of both purified recycled aromatic dicarboxylic acid (rCA) and purified aromatic dicarboxylic acid (vCA),

### FIELD AND BACKGROUND OF THE INVENTION

The present invention relates to a process and apparatus for production of both recycle and “virgin” aromatic carboxylic acids, particularly aromatic dicarboxylic acids.

A number of processes are known for the production of aromatic carboxylic acids, such as terephthalic acid, isophthalic acid and naphthalene dicarboxylic acid. These products themselves are useful for production of polymers. Terephthalic acid, for example, may be polymerised with ethylene glycol to produce polyethylene terephthalate, commonly known as PET.

The recycling of polymers generally, and polyesters such as PET in particular, is desirable for environmental reasons. Current techniques allow colourless, transparent poly(ethylene terephthalate) (PET) containers, such as bottles for soft drinks, to be recycled economically. In the recycling process, PET containers are sorted into different colours and baled. Baled containers made from clear and green PET are washed, flaked, and dried to form clean PET flakes. If necessary, the clean, clear PET flakes can be processed to remove any impurities (i.e., any component other than clean, clear PET flake and/or green/blue PET flake). The recycling of clean PET flakes can include depolymerization to break the ester bonds of the PET and reduce the polymer to its monomer components. Depolymerization can occur using several known reaction pathways, including, for example, via methanolysis or ethanolysis.

WO 2015/103178 describes a method for forming an aromatic diacid and/or an aromatic diacid precursor from a polyester-containing feedstock which comprises quantities of at least one secondary material, and wherein the at least one secondary material is not polyester. The method comprises contacting the polyester-containing feedstock with water or an alcohol to depolymerize the polyester and thereby form an aromatic diacid and/or an aromatic diacid precursor. The formed recycled terephthalic acid (rTA) may be utilised to form further polyethylene terephthalate polymers.

Recycled polymers typically have properties which are inferior to those of the corresponding virgin material. Consequently, commercial products are commonly made from a mixture of virgin and recycled material in order to ensure that the desired final properties are achieved. It is increasingly common for customers to wish to know the exact proportion of recycled material contained in such products. This can be difficult in conventional processes where the proportion of virgin and recycled material is not tracked continuously, such that even if the average proportion of recycled material is known the proportion in an individual batch is not.

### SUMMARY OF THE INVENTION

It is an objective to provide improved processes for the recycling of polyesters and other polymers produced from aromatic carboxylic acids. The present inventors have now found that both recycled aromatic carboxylic acid (rCA) and “virgin” aromatic carboxylic acid (vCA) can be advantageously co-produced from the same process in a way which permits simple control and measurement of the proportion or recycled material in a stream.

Thus, in a first aspect, the present invention provides a process for the production of both purified recycled aromatic dicarboxylic acid (rCA) and purified aromatic dicarboxylic acid (vCA), said process comprising:
a) producing a first stream comprising a recycled aromatic dicarboxylic acid by a first process, wherein the first process comprises the depolymerisation of a polymer feedstock comprising a polymer of said aromatic dicarboxylic acid in a first reaction system;
b) producing a second stream comprising the aromatic dicarboxylic acid by a second process wherein the second process comprises the oxidation of an aromatic hydrocarbon in a second reaction system;
c) purifying both the first stream and the second stream in a common purification system to thereby produce both purified recycled aromatic dicarboxylic acid (rCA) and purified aromatic dicarboxylic acid (vCA).

A particular feature of the present invention is that of both purified recycled aromatic dicarboxylic acid (rCA) and purified aromatic dicarboxylic acid (vCA) are produced from a process with a common purification system. Thus, both the stream comprising a recycled aromatic dicarboxylic acid produced in the first process and the stream comprising the aromatic dicarboxylic acid produced in the second process are purified in a common purification system.

A further advantage is that the present invention permits full control and also simple measurement of the proportion of rCA in the final product at all times.

### DETAILED DESCRIPTION OF THE INVENTION

For ease of reference, we may use the term “recycle” to refer to the aromatic dicarboxylic acid produced by the first process and the term “virgin” to refer to the aromatic dicarboxylic acid produced by the second process.

The process of the present invention has several advantages for the recycling of polymers of aromatic dicarboxylic acids.

Firstly, a “conventional” process for producing virgin aromatic dicarboxylic acid can be retrofitted by adding a process for producing an (“unpurified”) stream of the recycled aromatic dicarboxylic acid.

Secondly, a “combined” process as claimed provides significantly greater flexibility for the production of aromatic dicarboxylic acids. When sources of polymer of the aromatic dicarboxylic acid are plentiful (or “cheap”) then the overall process can be operated to produce recycled aromatic dicarboxylic acid (by the first process) as a higher percentage of the total production. In other circumstances the overall process can be operated to produce virgin aromatic dicarboxylic acid (by the second process) as a higher percentage of the total production or even with low or zero recycle content.

The purification step may also be operated according to such requirements. For example, if there is demand for a quantity of virgin aromatic dicarboxylic acid with low or zero recycle content, the process may be operated so that the first and second stream are purified separately, and in particular sequentially, in the purification system to separately produce purified virgin aromatic dicarboxylic acid (vCA) and purified recycled aromatic dicarboxylic acid (rCA).

In embodiments, the process can be operated to produce a mixture of recycled aromatic dicarboxylic acid and virgin aromatic dicarboxylic acid. In this case:
the purification steps may be operated sequentially, and at least a portion of the purified recycled aromatic dicarboxylic acid (rCA) blended with at least a portion of the purified aromatic dicarboxylic acid (vTA) after the purification step, or
the first and second streams, or at least portions of both, may be co-fed to the purification system and purified together to produce a blended product.

To assist in the ability to feed either one or both of the first stream and second stream to the purification system as required, particularly for separate (sequential) purification, one or more feed silos may be provided prior to the purification system. If it is then desired that a sequential purification takes place the product which is not being purified at a particular time can be collected and/or stored in a feed silo until its purification is required/scheduled. In preferred embodiments there is provided at least a first feed silo for recycled aromatic dicarboxylic acid produced in the first process and at least a second feed silo for aromatic dicarboxylic acid produced in the second process.

To further assist with the flexibility of the present invention there may also be provided one or more storage silos downstream of the purification system to which the purified recycled aromatic dicarboxylic acid (rCA) and purified aromatic dicarboxylic acid (vCA) are passed for storage after purification.

In preferred embodiments at least two storage silos may be provided downstream of the purification system. Where the first and second stream are fed sequentially to the purification system, for example, there is preferably provided at least a first storage silo utilised to store purified recycled aromatic dicarboxylic acid (rCA) and at least a second storage silo utilised to store purified aromatic dicarboxylic acid (vCA). If desired, the products from these silos can be blended, for example in a downstream blending or mixing step.

It may be noted that even where the purification system is operated to separately (sequentially) produce purified virgin aromatic dicarboxylic acid (vCA) and purified recycled aromatic dicarboxylic acid (rCA) that there can be a transition phase whilst the purification system is switched from purification of the first stream (i.e. production of purified recycled aromatic dicarboxylic acid (rCA)) to purification of the second stream (i.e. production of purified virgin aromatic dicarboxylic acid (vCA)), or vice versa. During the transition phase a mixed product stream comprising a mixture of purified recycled aromatic dicarboxylic acid and purified virgin aromatic dicarboxylic acid may be produced. One or more storage silos may be provided which are separate to any storage silos utilised to store purified recycled aromatic dicarboxylic acid (rCA) or to store purified aromatic dicarboxylic acid (vCA) and which are utilised to store mixed streams comprising both purified recycled aromatic dicarboxylic acid (rCA) and purified aromatic dicarboxylic acid (vCA).

The aromatic dicarboxylic acid may be any suitable aromatic dicarboxylic acid. (For avoidance of doubt, the first stream and the second stream generally comprise the same aromatic dicarboxylic acid i.e. the recycled aromatic dicarboxylic acid produced by the first process and the aromatic dicarboxylic acid produced by the second process are the same aromatic dicarboxylic acid.)

In preferred embodiments the aromatic dicarboxylic acid may be a benzene dicarboxylic acid, a naphthalene dicarboxylic acid or a furan dicarboxylic acid. Particularly preferably, the aromatic dicarboxylic acid is benzene dicarboxylic acid. Among these, isophthalic acid (benzene-1,3-dicarboxylic acid) and terephthalic acid (benzene-1,4-dicarboxylic acid) are particularly preferred.

Most preferably, the aromatic dicarboxylic acid is terephthalic acid. In this case, a particularly preferred embodiment provides a process for the production of both purified recycled terephthalic acid (rTA) and purified terephthalic acid (vTA), said process comprising:
a) producing a first stream comprising a recycled terephthalic acid by a first process, wherein the first process comprises the depolymerisation of a polymer feedstock comprising a polymer of terephthalic acid in a first reaction system
b) producing a second stream comprising terephthalic acid by a second process, wherein the second process comprises the oxidation of an aromatic hydrocarbon in a second reaction system;
c) purifying both the first stream and the second stream in a common purification system to thereby produce both purified recycled terephthalic acid (rTA) and purified terephthalic acid (vTA).

The polymer feedstock for the first process may, in general, be any suitable polymer feedstock comprising a polymer of the aromatic dicarboxylic acid. The polymer containing feedstock is typically a polyester (comprising the aromatic carboxylic acid). The polyester may, for example, be selected from the group consisting of polyethylene terephthalate (PET), polyethylene terephthalate glycol-modified (PETG), polyethylene naphthalate (PEN), polybutylene terephthalate (PBT), polypropylene-terephthalate (PPT), polyethylene-furanoate (PEF), and combinations thereof. In the case of the preferred embodiments, where the aromatic dicarboxylic acid is terephthalic acid or other benzene dicarboxylic acid, then the polymer feedstock is preferably a polyalkylene terephthalate, and most preferably polyethylene terephthalate (PET).

The first process may comprise any suitable reaction or reactions which can depolymerise the polymer feedstock and produce a first stream of the aromatic dicarboxylic acid. Suitable depolymerisation reactions may include alkanolysis, hydrolysis or glycolysis reactions, for example. Combinations of reactions may also be used, such as alkanolysis/hydrolysis, glycolysis/hydrolysis or even glycolysis/alkanolysis/hydrolysis. Other depolymerisation processes/reactions, such as salt formation followed by acidification can also be used. Where the feedstock is a polyester, for example, the preferred first process may comprise a hydrolysis or alkanolysis reaction. Particularly preferred methods include those described, for example, in WO 2015/103178 already noted, WO 2021257920 or U.S. Pat. No. 7,462,649.

The depolymerisation generally uses a suitable depolymerisation catalyst. Suitable catalysts include zinc chloride, zinc acetate, magnesium chloride, magnesium acetate, ammonium chloride, boron trifluoride, boron trichloride, boron tribromide, titanium chloride, sodium acetate, lithium acetate, manganese acetate, cobalt acetate, palladium acetate, copper acetate or titanium oxyacetylacetonate.

The depolymerisation reaction generally takes place at an elevated temperature, such as in the range 100-350° C., and elevated pressure, such as in the range of 5-60 bar, as known in the art.

Preferred methods for the first process include alkanolysis using a C₁-C₆ alcohol, especially using methanol, ethanol, propanol, isopropanol, or combinations thereof, and most preferably using methanol. The reaction may comprise use of an acid or acid precursor. Such processes, which are advantageous when water is present, are described, for example, in WO 2021/257920. Preferred acids, when used, are organic acids and mineral acids. Most preferred are organic acids, and in particular at least one organic acid selected from C₂-C₇ alkyl carboxylic acids (by which is meant acids of structure (C₁-C₆ alkyl)-COOH), aryl carboxylic acids, citric acid and fumaric acid. Most preferred at least one C₂-C₇ carboxylic acid, and particularly acetic acid.

This first process may also comprise one or more further steps or reactions (i.e. other than the depolymerisation reaction or reactions) as conventionally known in the art. Examples include steps or reactions to purify the polymer feedstock, for example to remove impurities, and conventional separation steps to separate aromatic dicarboxylic acid from unreacted polymer feedstock.

The second process comprises any suitable reaction or reactions for the oxidation of an aromatic hydrocarbon to produce the aromatic dicarboxylic acid. By “aromatic hydrocarbon” in this step is generally meant a monomer rather than a polymer. In preferred embodiments the aromatic hydrocarbon may be a dimethyl benzene, dimethyl naphthalene or a dimethyl furan, for example.

As a specific example, where the aromatic dicarboxylic acid is terephthalic acid then the aromatic hydrocarbon is usually 1,4-dimethylbenzene (para-xylene). As another specific example, where the aromatic dicarboxylic acid is isophthalic acid then the aromatic hydrocarbon is usually 1,3-dimethylbenzene (meta-xylene).

Production of such compounds by oxidation of aromatic hydrocarbons is well known, and in the present invention may be operated by any suitable route. Reference may be made, for example, to U.S. Pat. Nos. 9,394,223, 8,173,834 or 5,723,656.

The second process may also comprise one or more further steps or reactions (i.e. other than the oxidation reaction) as conventionally known in the art. Examples include conventional separation steps to separate aromatic dicarboxylic acid from unreacted aromatic hydrocarbon.

The purification of aromatic dicarboxylic acids in general terms is well-known in the art, such as described in, for example, the references already noted for the individual first and second processes, or U.S. Pat. No. 9,422,219.

The purification in the common purification system in the present invention, particularly when applied to purification of benzene dicarboxylic acid, and most specifically to purification of terephthalic acid, may comprise the following sequential steps:
i) Dissolving the aromatic dicarboxylic acid (either rCA or vCA or a mixture) in water at elevated temperature and pressure,
ii) Passing the solution to one or more crystallisers connected in series,
iii) Separating solid aromatic dicarboxylic acid from the residual solution in a solid/liquid separation step and/or by a washing step, and
iv) A drying step.

The above purification steps, when used, are generally applied independently of whether purifying the first stream, the second stream or a mixture.

In particular, although the first stream may contain different impurities than the second stream, the typical impurities in the first stream are all expected to be more soluble than the recycled aromatic dicarboxylic acid in water, and hence are still effectively removed in the purification steps. For example, in the preparation of recycled terephthalic acid, typical impurities include residual monomethyl terephthalate (MMT), dimethyl terephthalate (DMT), isophthalic acid (IA), its mono-and dimethyl esters (MMI and DMI) and cyclohexane dimethanol (CHDM), all of which are more soluble than terephthalic acid. Hence all of these impurities will be removed to acceptable levels without additional treatments.

Nevertheless, in some embodiments purification steps may be provided which are applied only when one or other of the first or second stream is being purified (either singularly or with the other stream). For example, in some embodiments a hydrogenation step may be applied when purifying recycled aromatic dicarboxylic acid, either when purifying the first stream “only” or purifying the first and second stream (or portions thereof) together. This can, for example, be utilised if it is desired to improve the colour of the purified recycled aromatic dicarboxylic acid. Hydrogenation of such streams is described, for example, in U.S. Pat. Nos. 5,095,145 or 5,473,102. However, we have found that in the present invention a hydrogenation step is not essential when the first rCA stream is being purified. Accordingly in one embodiment of the invention the first rCA stream is purified separately without a hydrogenation step. In another embodiment of the invention, when a mixed stream of rCA and vCA is purified, the amount of hydrogen used in the hydrogenation reactor is no more than that required to hydrogenate the vCA alone.

A further step in the above purification process, which may be applied when the first rCA stream is being purified (either singularly or with the second stream), is a step to remove organic impurities including methanol. Methanol is formed by the hydrolysis of monomethyl and/or dimethyl terephthalate (MMT and DMT) when the aromatic dicarboxylic acid is dissolved in water. It is therefore present in any vapour effluents released, particularly during the solid/liquid separation (recrystallisation) step (iii) and the drying step (iv). If not removed or at least significantly reduced in amount, methanol may contribute to problematic emissions of volatile organic compounds (VOCs).

This further step to remove organic impurities may involve:
separating vapour effluent released during any of steps (i) to (iv);
scrubbing the vapour effluent to form a scrubber effluent;
treating the scrubber effluent vapour to form a liquid treated scrubber effluent and a gaseous treated scrubber effluent; and
removing a least a portion of organic impurities from the liquid treated scrubber effluent, optionally by recovering and/or decomposing said organic impurities.

The scrubber effluent may optionally be treated to form a liquid treated scrubber effluent and a vapour treated scrubber effluent by condensing it in at least one heat exchanger. In such a case the gaseous treated scrubber effluent may additionally be thermally oxidised. Alternatively, the scrubber effluent may optionally be treated by scrubbing with a caustic substance to form a liquid treated scrubber effluent and a vapour treated scrubber effluent. In such a case the gaseous treated scrubber effluent may additionally be cooled and thermally oxidised. A further alternative is to treat the scrubber effluent by heating and then oxidising it in a thermal oxidizer to form the liquid treated scrubber effluent and a vapour treated scrubber effluent.

Use of the above methanol removal step permits the use of rCA containing up to 10 wt % (solid) of MMT and DMT in total. Further details of the methanol removal step can be found in U.S. Pat. No. 10,399,921.

In some embodiments, it may be possible for there to be other steps which are upstream of the purification system (or what is normally considered to be the purification system) but which are applied to the products from both the first and second process and which may be combined in a common step/system for the process of the present invention. For example, in some processes both the first reaction and the second reaction may comprise hydrolysis steps/systems and/or drying steps/systems, and in some embodiments these may be combined i.e. a common hydrolysis step may be present and/or a common drying step may be present. As a further example, where rCA and vCA are both separately stored in silos in a dried form, both would normally be reslurried prior to feeding to the purification system. The reslurrying of both may be performed in a common reslurrying drum.

In a further aspect, there is also provided an apparatus suitable for operating the process of the first aspect, and in particular for the production of both purified recycled aromatic dicarboxylic acid (rCA) and purified aromatic dicarboxylic acid (vCA), said apparatus comprising:
a) a first reaction system for producing a first stream comprising recycled aromatic dicarboxylic acid, wherein the first reaction system comprises a depolymerisation reactor for depolymerisation of a polymer feedstock comprising a polymer of said aromatic dicarboxylic acid;
b) a second reaction system for producing a second stream comprising aromatic dicarboxylic acid, wherein the second reaction system comprises an oxidation reactor for oxidation of an aromatic hydrocarbon;
c) a purification system, downstream of the first and second reaction systems and fluidly connected to both, for purifying the first stream and the second stream to produce both purified recycled aromatic dicarboxylic acid (rCA) and purified aromatic dicarboxylic acid (vCA).

The preferred embodiments of the apparatus are as described already for the preferred process/process steps in the first aspect.

For example, the first reaction system may comprise one or more reactors for performing alkanolysis, hydrolysis and/or glycolysis reactions i.e. the first reaction system may comprise one or more of an alkanolysis reactor, a hydrolysis reactor and a glycolysis reactor.

As another example, the apparatus may be provided with one or more feed silos for one or both of the first steam and the second stream, said one or more feed silos being located downstream of the respective reaction system and upstream of the purification system. For example, there may be provided at least a first feed silo for the first stream and at least a second feed silo for the second stream.

In other embodiments, the apparatus may comprise a mixing system wherein the first and second stream, or at least portions of both, are mixed prior to being co-fed to the purification system.

The apparatus may comprise one or more storage silos downstream of the purification system to which the purified recycled aromatic dicarboxylic acid (rCA) and purified aromatic dicarboxylic acid (vCA) are passed for storage after purification. For example, there may be provided at least a first storage silo utilised to store recycled aromatic dicarboxylic acid (rCA) and at least a second storage silo utilised to store purified aromatic dicarboxylic acid (vCA).

The apparatus may comprise a blending system downstream of the purification system and in which at least a portion of the purified recycled aromatic dicarboxylic acid (rCA) obtained is blended with at least a portion of the purified aromatic dicarboxylic acid (vCA).

In addition, the apparatus may comprise a polymer preparation unit upstream of the depolymerisation reactor. This may be used for treating the polymer feedstock prior to the depolymerisation reactor. Such a unit may be considered as part of the first reaction system, or may be upstream thereof.

In yet a further aspect, the purified recycled aromatic dicarboxylic acid (rCA) and purified aromatic dicarboxylic acid (vCA) produced may be utilised to produce a polymer of the aromatic dicarboxylic acid. Thus, in this further aspect there is provided a method for the production of a polymer of an aromatic dicarboxylic acid, which process comprises:
i) producing both purified recycled aromatic dicarboxylic acid (rCA) and purified aromatic dicarboxylic acid (vCA) by the process of the first aspect, and
ii) polymerising the purified recycled aromatic dicarboxylic acid (rCA) and purified aromatic dicarboxylic acid (vCA).

The preferred embodiments of this aspect are as provided for the first aspect. In particular, the aromatic dicarboxylic acid is preferably a benzene carboxylic acid, a naphthalene carboxylic acid or a furan carboxylic acid. Particularly preferably, the aromatic dicarboxylic acid is benzene dicarboxylic acid. Among these, isophthalic acid (benzene-1,3-dicarboxylic acid) and terephthalic acid (benzene-1,4-dicarboxylic acid) are particularly preferred. Most preferably, the aromatic carboxylic acid is terephthalic acid.

The polymer product in this aspect is any suitable polymer of the aromatic dicarboxylic acid, but in preferred embodiments is a polyester.

In one embodiment, the polymerisation of step (ii) may take place by polymerising a mixture comprising both the purified recycled aromatic dicarboxylic acid (rCA) and purified aromatic dicarboxylic acid (vCA), to thereby produce a polymer comprising recycled aromatic dicarboxylic acid.

In other embodiments purified recycled aromatic dicarboxylic acid (rCA) and purified aromatic dicarboxylic acid (vCA) streams may be polymerised separately to produce separate “virgin” and “recycled” polymer products (“vP” and “rP”). In a particularly preferred embodiment purified recycled aromatic dicarboxylic acid (rCA) and purified aromatic dicarboxylic acid (vCA) are polymerised separately, and subsequently at least a portion of a polymer obtained from polymerisation of purified recycled aromatic dicarboxylic acid is blended with at least a portion of a polymer obtained from polymerisation of purified aromatic dicarboxylic acid.

The present invention will be illustrated with respect to the following Example.

### EXAMPLE

The process of the Example produces recycled and virgin purified terephthalic acid, and in particular comprises:
A first process configured to depolymerise polyethylene terephthalate. The process includes trans-esterification then hydrolysis to yield a crude recycled terephthalic acid stream and a methanol stream which is recycled to the reactor. The crude recycled terephthalic acid is dissolved in water to remove methanol and other impurities, and then crystalised and dried to remove the water (and any residual methanol), before being passed to a first feed silo. The first feed silo has a capacity of 2000 units of the recycled terephthalic acid. Downstream of the first feed silo is a first reslurry tank. The crude recycled terephthalic acid is produced continuously at a rate of 10 units/hour.
A second process configured to oxidise paraxylene to yield a crude virgin terephthalic acid stream. The crude virgin terephthalic acid, which is in a dried, solid state, is passed to a second reslurry tank, optionally via a second feed silo as discussed below. The second feed silo has a capacity of 2500 units of the virgin terephthalic acid. The crude virgin terephthalic acid is produced continuously at a rate of 40 units/hour.
A purification system in which a slurry of (virgin or recycle) terephthalic acid is recrystallised from water in a series of crystallisers, subject to a solid-liquid separation and then dried to yield purified (virgin or recycle) terephthalic acid. The purification system has a capacity of 50 units/hour.
Downstream of the purification system there are provided a first storage silo for purified recycled terephthalic acid (rTA), a second storage silo for purified virgin terephthalic acid (vTA) and a third storage silo for material which comprises both rTA and vTA.

During all phases of operation both the first process and second process are operated continuously to produce the respective crude virgin and recycle terephthalic acid streams.

In the present Example, during a first phase the crude recycled terephthalic acid from the first process is passed to the first feed silo. At the same time, 50 units/hour of crude virgin terephthalic acid from the second process is passed to the second slurry tank, and from there to the purification section to produce purified vTA. In particular, the crude virgin terephthalic acid passed to the second slurry tank at this stage comprises 40 units/hour of crude virgin terephthalic acid which is being continuously produced by the second process, supplemented by 10 units/hour of crude virgin terephthalic acid from the second feed silo where it had been previously collected. (This is discussed further below.)

During this first phase of operation, the purified vTA is passed to the second storage silo where it is stored until required for further use, for example being passed to a polymerisation process, either “directly” if available on site, or after transport/shipping.

This first phase of operation is continued for approximately 8 days from its initiation, during which time 1920 units of crude recycled terephthalic acid from the first process is collected in the first storage silo, whilst 9600 units of crude virgin terephthalic acid is purified to give the same amount of purified virgin terephthalic acid (vTA).

After 8 days, the first feed silo is nearly full, and so the feeds to the second reslurry tank are both stopped, and the stream of crude virgin terephthalic acid obtained from the second process is redirected to the second feed silo, and collected therein.

At the start of this second phase the second feed silo still comprises about 500 units of crude virgin terephthalic acid, which is maintained as a buffer, and to which is then added 40 units/hour from the continuous production of the second process.

During this second phase the feed to the purification section is switched so as to be fed from the first feed silo rather than the second feed silo. In particular, the first feed silo passes the crude recycled terephthalic acid therefrom at a rate of 50 units/hour to the first reslurry tank, and from there to the purification section to produce purified rTA. (It should be noted that the feed to the first reslurry tank could be started before the first phase is finished so that a slurry is present in the first reslurry tank in preparation for the second phase starting.)

During the initial stages after switching the feed to the purification section (from the second reslurry tank to the first reslurry tank), the stream exiting the purification section initially still comprises essentially purified vTA, which then transitions to a stream comprising essentially rTA. This may take place, for example, over a period of about 1-4 hours. During this time, or at least between the times when purified vTA and purified rTA of sufficient purity are obtained, the stream exiting the purification section is passed to the third storage silo. Once sufficiently pure purified rTA is being obtained, this product is then passed to the first storage silo.

(The purified rTA is also stored until required for further use, for example being passed to a polymerisation process, either “directly” if available on site, or after transport/shipping.)

This second phase is continued for approximately 48 hours, during which time there are fed to the purification section the 1920 units of crude recycled terephthalic acid present in the first feed silo at the start of this second phase, as well as 480 units of production from the first process during this time.

Also during this time, approximately 1920 units of crude virgin terephthalic acid is collected in the second feed silo. At the end of this second phase, the first feed silo is essentially empty, whilst the second silo contains approximately 2420 units of crude virgin terephthalic acid.

The operation is then switched back to the initial “operation” (first phase) already described. In particular, the feed from the first silo to the first reslurry tank is stopped, the feeds to the second reslurry tank are both reopened (40 units/hour of crude virgin terephthalic acid which is being continuously produced by the second process and 10 units/hour of crude virgin terephthalic acid from the second feed silo), and the feed to the purification section is switched so as to be fed from the second feed silo (via the second reslurry tank). 50 units/hour of crude virgin terephthalic acid are therefore again passed from the second process to the second slurry tank, and from there to the purification section to produce purified vTA. (It should be noted that the feeds to the second reslurry tank could be started before the second phase is finished so that a slurry is present in the second reslurry tank in preparation for the first phase restarting.) (It should also be noted that although in this Example we refer to the 50 units/hour to the second reslurry tank comprising 40 units/hour of crude virgin terephthalic acid which is being continuously produced by the second process and 10 units/hour of crude virgin terephthalic acid from the second fed silo, in fact the 40 units/hour being produced in the second process need not be passed “directly” to the second reslurry tank, and could continue to be passed to the second feed with 50 units/hour being passed from the second feed silo to the second reslurry tank. This would still have the effect of reducing the inventory in the second feed silo. This would have the advantage that the stream from the second process is always passed to the second feed silo, and does not need to be redirected during changeovers.)

It will be noted that, during the initial stages after switching the feed to the purification section, the stream exiting the purification section initially still comprises essentially purified rTA, which then transitions to a stream comprising essentially vTA. This may take place, for example, over a period of about 1-4 hours. During this time, or at least between the times when purified rTA and purified vTA of sufficient purity are obtained, the stream exiting the purification section is passed to the third storage silo. Once sufficiently pure purified vTA is being obtained, this product is then passed to the second storage silo as previously described.

In some embodiments a hydrogenation step may be performed in the purification section to remove impurities and improve the colour of the purified vTA, the purified rTA, or both (i.e. the hydrogenation step may be performed during the first phase, the second phase, or during both phases). For example, depending on the source of the polyethylene terephthalate fed to the first process, a hydrogenation step may be performed in the purification section during the second phase to remove impurities and improve the colour of the purified rTA. If hydrogenation is only normally applied in the second phase, but not in the first phase, then hydrogenation would typically also be performed during the switch i.e. where a mixture of products is being formed.

In this Example, the process can switch between the phases as required to enable all of the production to be purified and kept separate.

If desired, nevertheless the production could be mixed after purification, or a mixture of the first and second streams could be passed to the purification section.

1. A process for producing both purified recycled aromatic dicarboxylic acid (rCA) and purified aromatic dicarboxylic acid (vCA), said process comprising:
a) Producing a first stream comprising a recycled aromatic dicarboxylic acid by a first process, wherein the first process comprises the depolymerisation of a polymer feedstock comprising a polymer of said aromatic dicarboxylic acid in a first reaction system
b) Producing a second stream comprising the aromatic dicarboxylic acid by a second process, wherein the second process comprises the oxidation of an aromatic hydrocarbon in a second reaction system;
c) Purifying both the first stream and the second stream in a common purification system to thereby produce both purified recycled aromatic dicarboxylic acid (rCA) and purified aromatic dicarboxylic acid (vCA).

2. The process according to claim 1 wherein the aromatic carboxylic acid is a benzene dicarboxylic acid, a naphthalene dicarboxylic acid or a furan dicarboxylic acid.

3. The process according to claim 1 wherein the aromatic dicarboxylic acid is terephthalic acid.

4. The process according to claim 1 wherein the first and second stream are purified sequentially in the purification system to sequentially produce the purified recycled aromatic dicarboxylic acid (rCA) and purified aromatic dicarboxylic acid (vCA).

5. The process according to claim 4 wherein subsequently at least a portion of the purified recycled aromatic dicarboxylic acid (rCA) is blended with at least a portion of the purified aromatic dicarboxylic acid (vTA).

6. The process according to claim 1 wherein the first and second stream, or at least portions of both the first and second stream, are co-fed to the purification system.

7. The process according to claim 1 wherein there one or more feed silos for one or both of the first steam and the second stream are provided and located prior to the purification system.

8. The process according to claim 7 wherein at least a first feed silo is provided for the first stream and at least a second feed silo is provided for the second stream.

9. The process according to claim 1 further comprising one or more storage silos downstream of the purification system to which the purified recycled aromatic dicarboxylic acid (rCA) and purified aromatic dicarboxylic acid (vCA) are passed for storage after purification.

10. The process according to claim 9 wherein the first and second stream are fed sequentially to the purification system, and at least a first storage silo is provided and utilised to store purified recycled aromatic dicarboxylic acid (rCA) and at least a second storage silo is provided and utilised to store purified aromatic dicarboxylic acid (vCA).

11. The process according to claim 1, wherein the purification step (c) comprises the steps of:
(i) dissolving the aromatic dicarboxylic acid (either rCA or vCA or a mixture thereof) in water at elevated temperature and pressure,
(ii) passing the solution to one or more crystallisers connected in series,
(iii) separating solid aromatic dicarboxylic acid from the residual solution in a solid/liquid separation step and/or by a washing step, and
(iv) a drying step.

12. The process according to claim 11, further comprising the steps of
separating a vapour effluent released during any of steps (i) to (iv);
scrubbing the vapour effluent to form a scrubber effluent;
treating the scrubber effluent to form a liquid treated scrubber effluent and a gaseous treated scrubber effluent; and
removing a least a portion of organic impurities from the liquid treated scrubber effluent, optionally by recovering and/or decomposing said organic impurities.

13. The process according to claim 1, wherein either the first stream (rCA) is purified separately without a hydrogenation step, or a mixed stream of rCA and vCA is purified and an amount of hydrogen used in the hydrogenation reactor is no more than an amount of hydrogen required to hydrogenate the vCA alone.

14. Apparatus for the production of both purified recycled aromatic dicarboxylic acid (rCA) and purified aromatic dicarboxylic acid (vCA), said apparatus comprising:
a) a first reaction system for producing a first stream comprising recycled aromatic dicarboxylic acid, wherein the first reaction system comprises a depolymerisation reactor for depolymerisation of a polymer feedstock comprising a polymer of said aromatic dicarboxylic acid;
b) a second reaction system for producing a second stream comprising the aromatic dicarboxylic acid, wherein the second reaction system comprises an oxidation reactor for oxidation of an aromatic hydrocarbon;
c) a purification system, downstream of the first and second reaction systems and fluidly connected to both the first and second reaction systems, for purifying the first stream and the second stream to produce both purified recycled aromatic dicarboxylic acid (rCA) and purified aromatic dicarboxylic acid (vCA).

15. The apparatus according to claim 14 which comprises a blending system downstream of the purification system and in which at least a portion of the purified recycled aromatic dicarboxylic acid (rCA) obtained is blended with at least a portion of the purified aromatic dicarboxylic acid (vCA).

16. The apparatus according to claim 14 comprising a mixing system wherein the first and second stream, or at least portions of both the first and second stream, are mixed prior to being co-fed to the purification system.

17. The apparatus according to claim 14 further comprising a polymer preparation unit upstream of the depolymerisation reactor for treating the polymer feedstock prior to the depolymerisation reactor.

18. A method for producing a polymer of an aromatic dicarboxylic acid, which method comprises:
i) producing both purified recycled aromatic dicarboxylic acid (rCA) and purified aromatic dicarboxylic acid (vCA) by the process of claim 1, and
ii) polymerising the purified recycled aromatic dicarboxylic acid (rCA) and purified aromatic dicarboxylic acid (vCA).

19. The method according to claim 18 wherein the aromatic dicarboxylic acid is a benzene dicarboxylic acid, a naphthalene dicarboxylic acid or a furan dicarboxylic acid.

20. The method according to claim 18 wherein the aromatic dicarboxylic acid is terephthalic acid.
